(19) United States
(12) Patent Application Publication
(10) Pub. No.: US 2021/0079050 A1
(43) Pub. Date: Mar. 18, 2021

(54) COMPOSITIONS AND METHODS FOR DELIVERY AND EXPRESSION OF SMALL INHIBITORY PEPTIDES AND USE THEREOF

(71) Applicant: CARMEL HAIFA UNIVERSITY ECONOMIC CORPORATION LTD., Haifa (IL)

(72) Inventors: Kobi ROSENBLUM, Zichron Yaacov (IL); Efrat EDRY, Kiryat Ata (IL)

(21) Appl. No.: 16/971,234

(22) PCT Filed: Feb. 20, 2019
(86) PCT No.: PCT/IL2019/050202

§ 371 Date: Aug. 19, 2020

Related U.S. Application Data
(60) Provisional application No. 62/632,505, filed on Feb. 20, 2018.

(51) Int. Cl. C07K 14/47; A61P 25/28; C12N 15/113

(52) U.S. Cl. CPC C07K 14/4702; A61P 25/28; A61K 38/00; C07K 2319/02; C07K 2317/24; C12N 15/113

(57) ABSTRACT

The present invention is directed to, inter alia, a chimeric polynucleotide made up of a first polynucleotide encoding a specific cell predominantly expressed protein precursor and a second polynucleotide encoding a modulating-peptide. Further provided are methods for specific delivery of small modulating peptides to specific cells. Also provided are methods for treating cell-specific-associated diseases, including but not limited to, muscular, neural, hepatic and pancreatic disease, in a subject in need thereof.

### CROSS REFERENCE TO RELATED APPLICATIONS

This application claims the benefit of priority of U.S. Provisional Patent Application No. 62/632,505, filed Feb. 20, 2018, the contents of which are all incorporated herein by reference in their entirety.

### FIELD OF INVENTION

The present invention is directed to, inter alia, expression and delivery of small inhibitory peptides, and use thereof such as in the therapy of cell-specific-associated diseases.

### BACKGROUND OF INVENTION

Bioactive peptides are present in all organisms and play diverse roles in their physiology. Peptides are defined here as biologically active molecules composed of 2 to 50 amino acids coupled to each other through an amide or disulfide bond. In the late 1990s and 2000s the pharmaceutical industry has expanded research regarding peptides as therapeutic agents, owing to their many advantages. These advantages include high potency, high selectivity, relative lack of toxicity, and predictable metabolism. However, peptides suffer from disadvantages, which include chemical and physical instability, high susceptibility to degradation by approximately 600 proteases in the human body (present in tissues and blood circulation), susceptibility to hydrolysis and oxidation, tendency for aggregation, short half-life and fast elimination, limited bioavailability due to their low membrane permeability, and consequently, the inability to administer them orally.

### SUMMARY OF THE INVENTION

The present invention, in some embodiments thereof, relates to a chimeric polynucleotide encoding a protein precursor, a polynucleotide encoding a protease/convertase and a polynucleotide encoding a modulating-peptide of 2-50 amino acids, and more particularly, but not exclusively, to methods for specifically modifying specific cell activity comprising administering such compositions to specific cells activity comprising administrating such compositions to specific cells.

According to one aspect, there is provided a chimeric polynucleotide molecule comprising: a first polynucleotide encoding a propeptide domain of a first polypeptide; and a second polynucleotide encoding a second polypeptide of 2-50 amino acids having a modulating activity, wherein the propeptide domain is a specific cell predominantly expressed protein precursor, wherein the first polypeptide and said second polypeptide are not derived from the same gene and are operably linked.

In some embodiments, the propeptide domain comprises a signal peptide sequence and a protease/convertase motif.

In some embodiments, the signal peptide sequence and the protease/convertase motif are endogenous to the propeptide.

In some embodiments, the modulating peptide is an endogenous peptide. In some embodiments, the modulating peptide is an exogenous peptide. In some embodiments, the modulating peptide is a synthetic peptide.

In some embodiments, the propeptide domain is encoded by a polynucleotide sequence as set forth in SEQ ID NO:1 or SEQ ID NO: 2.

In some embodiments, a delivery vector for expression of the disclosed chimeric polynucleotide is provided.

In some embodiments, the delivery vector comprises a promoter polynucleotide for driving the expression of the disclosed chimeric polynucleotide predominantly in a specific cell.

In some embodiments, the specific cell is a neural cell.

In some embodiments, the specific cell is a muscle cell.

In some embodiments, the specific cell is a liver cell.

In some embodiments, the specific cell is a pancreas cell.

In some embodiments, the first polynucleotide sequence encoding the propeptide domain of the first polypeptide is selected from the group consisting of neurotrophic factors.

In some embodiments, the first polynucleotide sequence encoding the propeptide domain of the first polypeptide is selected from the group consisting of procollagens.

In some embodiments, the first polynucleotide sequence encoding the propeptide domain of the first polypeptide is derived from factor X.

In some embodiments, the first polynucleotide sequence encoding the propeptide domain of the first polypeptide is selected from the group consisting of: proinsulin, proglucagon, pro somatostatin, and proghrelin.

In some embodiments, a delivery vector for use in a cell population selected from the group consisting of: sensory neurons, motor neurons, interneurons, neurons of the brain, astrocytes, microglia, ependymal cells, oligodendrocytes, Schwann cells, satellite cells, enteric glial cells, olfactory cells, and sheathing cells, is provided.

In some embodiments, a delivery vector for use in a cell population selected from the group consisting of: red skeletal muscle cell, white skeletal muscle cell, intermediate muscle cell, nuclear bag cell, nuclear chain cell, satellite cell, heart muscle cell, nodal heart muscle cell, Purkinje fiber cell, smooth muscle cell, and myoepithelial cell, is provided.

In some embodiments, a delivery vector for use in a cell population selected from the group consisting of: hepatocyte, Kupffer cell, and liver stellate cell, is provided.

In some embodiments, a delivery vector for use in a cell population selected from the group consisting of: Alpha cell, Beta cell, Delta cell, Gamma cell (i.e., PP cell), and Epsilon cell, is provided.

In some embodiments, a cell comprising the disclosed delivery vector, is provided.

In some embodiments, the cell is selected from the group consisting of: sensory neurons, motor neurons, interneurons, neurons of the brain, astrocytes, microglia, ependymal cells, oligodendrocytes, Schwann cells, satellite cells, enteric glial cells, olfactory cells, and sheathing cells.

In some embodiments, the cell is selected from the group consisting of: red skeletal muscle cell, white skeletal muscle cell, intermediate muscle cell, nuclear bag cell, nuclear chain cell, satellite cell, heart muscle cell, nodal heart muscle cell, Purkinje fiber cell, smooth muscle cell, and myoepithelial cell.

In some embodiments, the cell is selected from the group consisting of: hepatocyte, Kupffer cell, and liver stellate cell.

In some embodiments, the cell is selected from the group consisting of: Alpha cell, Beta cell, Delta cell, Gamma cell, and Epsilon cell.

In some embodiments, a chimeric polypeptide encoded by any one of: the disclosed chimeric polynucleotide; or the disclosed delivery vector, is provided.

In some embodiments, a method for modulating a specific cell, comprising the step of contacting the specific cell with any one of: the disclosed chimeric polynucleotide; or the disclosed delivery vector; thereby modulating the specific cell, is provided.

In some embodiments, contacting the specific cell comprises the step of systemically administering to a subject in need thereof the chimeric polynucleotide, thereby producing post-translationally active modulating peptide only in a specific cell of the subject, thereby modulating only the specific cell of the subject.

In some embodiments, a method for treating cell-specific-associated disease or disorder in a subject in need thereof, the method comprising administering to the subject any one of: the disclosed chimeric polynucleotide; or the disclosed delivery vector; thereby treating cell-specific-associated disease in the subject, is provided

In some embodiments, the disease or disorder is selected from the group consisting of: degenerative disease, developmental disease or cancer disease.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a non-limiting schematic illustration of a vector of the disclosed invention comprising a lentivirus (LV) expressing the peptide construct (propeptide). The upper panel represents the viral genome expressing the peptide precursor and the green fluorescence protein (GFP) marker under the regulation of the ubiquitin promoter (UBI). IRES, Internal Ribosome Entry Site; WPRE, Woodchuck Hepatitis Virus (WHP) Posttranscriptional Regulatory Element; and cPPT, Central Polypurine tract. The lower panel represents the peptide precursor, which comprises the proBDNF domain, HA tag, TAT sequence and peptide of interest. The arrow represents proteolytic cleavage site.

FIGS. 2A-2L are fluorescent micrographs of viral infection of COS-7 cell lines. Transgene overexpression in brain-derived neurotrophic factor (BDNF)- or propeptide lentiviral (LV)-infected compared to untreated COS-7 cells. BDNF (control) or propeptide LV infected or untreated COS-7 cells were grown on PLL coated cover slips in a 12 wells plate to 50% confluency. On the day of the experiment cells were fixed with paraformaldehyde 4%, and then immunostained with anti-proBDNF 1:200 (2B, 2F and 2J), nuclei were stained by DAPI 1:5,000 (2D, 2H and 2L), infection was observed by GFP fluorescence (2C, 2G and 2K). (2A-2D) BDNF infected cells; (2E-2H) propeptide infected cells; and (2I-2L) control cells. Magnification x20 and scale bar=20 μm.

FIGS. 3A-3L are fluorescent micrographs of viral infection of 3T3/NIH cell lines. Transgene overexpression in BDNF- or propeptide lentiviral (LV)-infected compared to untreated 3T3/NIH. BDNF (control) or propeptide LV infected or untreated 3T3/NIH cells were grown on PLL coated cover slips in a 12 wells plate to 50% confluency. On the day of the experiment cells were fixed with paraformaldehyde 4%, and then immunostained with anti-proBDNF 1:200 (3B, 3F and 3J), nuclei were stained by DAPI 1:5,000 (3D, 3H and 3L), infection was observed by GFP fluorescence (3C, 3G and 3K). (3A-3D) BDNF infected cells; (3E-3H) propeptide infected cells; and (I-L) control cells. Magnification x20 and scale bar=20 μm.

FIGS. 4A-4L are fluorescent micrographs of viral infection of COS-7 cell lines. Transgene overexpression in BDNF- or propeptide lentiviral (LV)-infected compared to untreated COS-7 cells. BDNF (control) or propeptide LV infected or untreated COS-7 cells were grown on PLL coated cover slips in a 12 wells plate to 50% confluency. On the day of the experiment cells were fixed with paraformaldehyde 4%, and then immunostained with anti-HA-tag 1:200 (4B, 4F and 4J), nuclei were stained by DAPI 1:5,000 (4D, 4H and 4L), infection was observed by GFP fluorescence (4C, 4G and 4K). (4A-4D) BDNF infected cells; (4E-4H) propeptide infected cells; and (4I-4L) control cells. Magnification x20 and scale bar=20 μm.

FIGS. 5A-5L are fluorescent micrographs of viral infection of 3T3/NIH cell lines. Transgene overexpression in BDNF- or propeptide lentiviral (LV)-infected compared to untreated 3T3/NIH cells. BDNF (control) or propeptide LV infected or untreated 3T3/NIH cells were grown on PLL coated cover slips in a 12 wells plate to 50% confluency. On the day of the experiment cells were fixed with paraformaldehyde 4%, and then immunostained with anti-HA-tag 1:200 (5B, 5F and 5J), nuclei were stained by DAPI 1:5,000 (5D, 5H and 5L), infection was observed by GFP fluorescence (5C, 5G and 5K). (5A-5D) BDNF infected cells; (5E-5H) propeptide infected cells; and (5I-5L) control cells. Magnification x20 and scale bar=20 μm.

FIGS. 6A-6B are immunoblot images of lysates and media obtained from infected COS-7 and 3T3/NIH cells. The propeptide transgene undergoes BDNF-like processing in infected COS-7 and 3T3/NIH cells. 1×10⁵ COS-7 or 3T3/NIH cells were seeded in a 24 wells plate, infected with BDNF or propeptide vectors and grown to full confluency. Cells were passed to a 6 wells plate and grown to a full confluency again. The cells were harvested by RIPA buffer and the medium was collected, lyophilized and resuspended in 500 ml of RIPA buffer. (6A) Representative immunoblots from lysates of COS-7 cells infected with BDNF (upper panel) or propeptide virus (lower panel). (6B) Representative immunoblots from medium collected from BDNF or BPEP vectors infected COS-7 and 3T3/NIH cells.

FIGS. 7A-7B are an immunoblot image (7A) and a graph (7B) describing the effects of propeptide transduction on IκB degradation in Poly I:C transfected 3T3/NIH cells. (7A) Cells were seeded in a 24-wells plate, infected with propeptide vector or left untreated and grown to 100% confluency. Cells were then passed to a 6 well plate and transfected with 0.25 or 1 μg of Poly I:C for 18 hr. The cells were harvested by RIPA buffer and western blot analysis was performed. (7A) Representative Immunoblots; and (7B) IκB levels presented as the ratio between IκB and β-actin (N≥2) in the corresponding immunoblots. Results were normalized to untreated cells. Data presented as mean±SEM, * p<0.05.

FIGS. 8A-8E are a table, images and graphs describing the effect of vector mediated peptide delivery into CA1 which enhances spatial and learning memory. (8A) A table summarizing the set of semi-randomly selected distal start positions for acquisition training, with the platform being located in the SW quadrant. (8B) Illustration of the MWM arena. P: platform. (8C) Illustration of injection site. (8D) Propeptide (vector) were injected to mice which subsequently displayed improved Morris water maze (MWM) learning. Escape latency was measured across 5 days of MWM task and showed that propeptide injected mice acquired better spatial learning than the control group (GFP). (8E) Propeptide injected mice show a tendency for enhanced memory in Morris water maze probe test (day 4). Mice were trained using a standard Morris water maze paradigm and some of them were tested for spatial of platform location in the probe test (day 4). Results are presented as escape latency to platform location during the probe test. For both assays: Propeptide (n=10) and GFP (n=9); Error bars are mean±SEM. Statistical significance was examined using student's t-test; * p=0.08.

FIGS. 9A-9D are illustrations and graphs. (9A) Illustration showing a non-limiting fear conditioning (FC) protocol, such as was used herein to test the animals. (9B) An overview illustration of the contextual and cued fear conditioning test. (9C) A graph showing the cue test did not demonstrate any difference in freezing percentage in Bpep injected animals at tones or time interval (n≥30) compared to control group (n≥30). (9D) A vertical bar graph showing that freezing response following contextual FC 24 hr after conditioning revealed significant difference in freezing memory in Bpep injected animals compared to GFP controls. (Data represented as Mean±SEM. Student's t-test; *p<0.05).

FIGS. 10A-10C are a western-blot image (10A) and vertical bar graph (10B-10C) showing that virally mediated proBDNF-EPE expression results in inhibition of ERK1/2 nuclear translocation. COS7 cells were grown in full medium and then serum starved (16 h, 0.1%), pretreated either with LV expressing EPE peptide (10 μl) or untreated as control, and then either stimulated with tetradecanoylphorbol acetate (TPA; 200 nM or 500 nM, for 15 min) or left untreated (NT) as control. (10A) Nuclear ERK and cytosolic ERK protein levels were evaluated by western-blot. Immunoblots of ERK1/2 (cytosol and nuclear): Not infected—not treated (NI NT), Infected—not treated (BDNF NT), Not infected—200 nM (NI 200), Infected—200 nM (BDNF 200), Not infected—500 nM (NI 500), Infected—500 nM (BDNF 500). (10B) and (10C) are bar graphs showing inhibition of ERK1 or ERK2 nuclear translocation, respectively, which is expressed as the ratio between ERK nuclear levels and ERK cytosolic levels. Results were normalized to untreated samples.

FIGS. 11A-11C are a western-blot image (11A) and vertical bar graphs (11B-11C) showing that virally mediated proNGF-EPE expression results in inhibition of ERK1/2 nuclear translocation. COS7 cells were grown in full medium and then serum starved (16 h, 0.1%), pretreated either with LV expressing EPE peptide (10 μl) or untreated as control, and then either stimulated with tetradecanoylphorbol acetate (TPA; 200 nM or 500 nM, for 15 min) or left untreated (NT) as control. (11A) Nuclear ERK and cytosolic ERK protein levels were evaluated by western-blot. Immunoblots of ERK1/2 (cytosol and nuclear): Not infected—not treated (NI NT), Infected—not treated (NGF NT), Not infected—200 nM (NI 200), Infected—200 nM (NGF 200), Not infected-500 nM (NI 500), Infected—500 nM (NGF 500). (10B) and (10C) are bar graphs showing inhibition of ERK1 or ERK2 nuclear translocation, respectively, which is expressed as the ratio between ERK nuclear levels and ERK cytosolic levels. Results were normalized to untreated samples.

FIGS. 12A-12B are vertical bar graphs showing virally mediated proBDNF-EPE (12A) and proNGF-EPE (12B) expression results in inhibition of ERK1/2 nuclear translocation. proBDNF-EPE/proNGF-EPE LV infected or untreated COS-7 cells were grown on Poly L Lysine (PLL) coated cover slips in a 12-well plate until to 50% confluency. On the day of the experiment cells were fixed with paraformaldehyde 4%, and then immunostained with anti-ERK (1:100). Inhibition of ERK1/2 nuclear translocation is expressed as the ratio between ERK nuclear levels and ERK cytosolic levels.

### DETAILED DESCRIPTION OF THE INVENTION

The present invention, in some embodiments thereof, relates to a chimeric polynucleotide encoding a protein precursor and a modulating-peptide, and more particularly, but not exclusively, to methods for specifically modifying specific cell activity comprising administrating such compositions to specific cells.

In another embodiment, the present invention is further directed to a method for modulating specific cell activity, comprising administrating to the cell pharmaceutical composition of therapeutically effective amounts of small modulating peptides. In another embodiment, the present invention is further directed to a method for modulating specific cell activity, comprising the step of systemically administrating to a subject in need thereof a pharmaceutical composition in a therapeutically effective amount of small modulating peptides thereby modulating activity of only specific cells.

The present invention is based, in part, on the finding that post translation processing of a chimeric precursor protein delivered active small peptides predominantly to a specific target cell. As exemplified herein, processing neural-specific neurotrophic precursor proteins, which require a neural specific processing and delivering machinery, took place primarily in neural cells, and thus gave rise to active modulating peptides in these cells, in vivo. The invention is further based, in part, on the finding that by utilizing cell-specific post translational modification and processing of a precursor propeptide chimerically linked to modulating peptides, a targeted therapeutic response relevant for cell-specific-associated diseases can be achieved.

### Polynucleotides, Peptides and Vectors

As used herein, the term “polynucleotide” refers to a single or double stranded nucleic acid sequence which is isolated and provided in the form of an RNA sequence, a complementary polynucleotide sequence (cDNA), a genomic polynucleotide sequence and/or a chimeric polynucleotide sequences (e.g., a combination of the above).

According to some embodiments, the present invention is directed to the delivery and expression of a modulating peptide. In one embodiment, the modulating peptide is encoded by a polynucleotide. In one embodiment, the modulating peptide is encoded by a chimeric polynucleotide of the invention. In one embodiment, the modulating peptide is an endogenous peptide. In one embodiment, the modulating peptide is an exogenous peptide. In one embodiment, the modulating peptide is a synthetic peptide. In one embodiment, the modulating peptide comprises at least 2 amino acids, 2-5 amino acids, 4-8 amino acids, 7-15 amino acids, 14-25 amino acids, 24-35 amino acids and 34-50 amino acids.

As defined herein, “modulating activity” is an activity attributed to a modulating peptide. In one embodiment, a modulating peptide is an inhibitory peptide. In one embodiment, a modulating peptide is a stimulatory peptide. In some embodiments, a modulating peptide induces cell death. In one embodiment, a modulating peptide induces cell apoptosis. In one embodiment, a modulating peptide induces cell survival. In one embodiment, a modulating peptide enhances a signal transduction pathway in a target cell. In one embodiment, a modulating peptide blocks an intracellular cascade. In one embodiment, a modulating peptide blocks cell to cell communication. In one embodiment, a modulating peptide is a hormone. In one embodiment, a modulating peptide is a secreted peptide. In one embodiment, a modulating peptide is an antigen. In one embodiment, a modulating peptide is a toxin. In one embodiment, a modulating peptide inhibits protein-protein interaction. In one embodiment, a modulating peptide inhibits protein translocation from the cytosol into the nucleus, inhibits translocation from the nucleus to the cytosol, or both.

The terms “precursor” and “propeptide” are used herein interchangeably.

As defined herein, the term “specific cell predominantly expressed protein precursor” refers to any gene product (e.g., RNA or peptide) that is found primarily in a specific cell type compared to any other cell type. In some embodiments, specific cell predominantly expressed protein precursor refers to a gene product that is primarily secreted from a specific cell type compared to any other cell type. In some embodiments, specific cell predominantly expressed protein precursor refers to any gene product that is found primarily or primarily secreted from a specific cell of one subtype compared to any other specific cell of a different subtype originating from the same tissue. Non-limiting examples of specific cell types include: epithelial cells, hormone secreting cells, neural cells, metabolism and storage cells, adipocyte, hepatocyte, kidney cell, pancreatic cell, brush border cell, epidydimal cell, endothelial cell, chondrocyte, osteoblast/osteocyte, skeletal muscle cell, cardiomyocyte, smooth muscle cell, blood cell, immune cell, germ cell and nurse cell. As would be apparent to one skilled in the art, a specific cell type may include, but not limited to, progenitor cell, satellite cell, fully or partially differentiated cell, neoplastically transformed cell, and others.

In one embodiment, “specific-cell predominantly expressed mRNA” refers to an increased mRNA level in a specific cell. In some embodiments, increased mRNA level is a result of increased activation of a cell-specific gene's promoter sequence. In some embodiments, increased mRNA level is increased number of mRNA transcript molecules. In some embodiments, increased mRNA level is increased mRNA stability. In some embodiments, increased mRNA level is at least 2-fold, 5-fold, 10-fold, 50-fold, 100-fold, or 1,000-fold more in a specific cell type compared to any other cell type, and any value and range therebetween. In some embodiments, increased mRNA level is 2- to 5-fold, 4- to 10-fold, 8- to 50-fold, 40- to 150-fold, 100- to 500-fold, or 400- to 1,000-fold more in a specific cell type compared to any other cell type. Each possibility represents a separate embodiment of the invention. Methods of determining gene overexpression (i.e., increased mRNA levels) are well known to the skilled artisan, and include non-limiting examples, such as quantitative real-time RT-PCR, qualitative real-time RT-PCR, and others.

In some embodiments, “specific-cell predominantly expressed peptide” refers to an increased level of a translated peptide in a specific cell. In some embodiments, an increased level of a translated peptide refers to an increased level of the peptide in the producing cell. In some embodiments, increased levels of a translated peptide refer to increased levels of said peptide in the cytosol of the producing cell. In some embodiments, increased levels of a translated peptide refer to increased levels of the peptide in the secretory pathway of the producing cell. In some embodiments, increased levels of a translated peptide refer to increased levels of sorting the peptide to secretory vesicles. In some embodiments, increased levels of a translated peptide refer to increased secretion of the peptide from the producing cell. In some embodiments, increased levels of a translated peptide refer to a peptide primarily secreted from a specific cell type compared to any other cell type. In some embodiments, increased peptide level is at least 2-fold, 5-fold, 10-fold, 50-fold, 100-fold, or 1,000-fold more in a specific cell type compared to any other cell type, and any value and range therebetween. In some embodiments, increased peptide level is 2- to 5-fold, 4- to 10-fold, 8- to 50-fold, 40- to 150-fold, 100- to 500-fold, or 400- to 1,000-fold more in a specific cell type compared to any other cell type. Each possibility represents a separate embodiment of the invention. Methods of determining increased peptide production are well known to the skilled artisan, and include non-limiting examples, such as immunohistochemistry, immunocytochemistry, Enzyme-linked immunosorbent assay (ELISA, e.g., direct, indirect, ‘sandwich’, etc.), western-blot, dot-blot, and others.

In some embodiments, “specific-cell predominantly expressed peptide” refers to increased levels of properly processed peptide in a specific cell. In some embodiments, properly processed refers to proteolytic cleavage (such as that lack thereof hinders activity). In some embodiments, properly processed refers to removal of certain amino acids. In some embodiments, properly processed refers to an accurate folding. In another embodiment, accurate folding comprises formation of peptide secondary structures (e.g., α-helix, β-sheet, etc.), disulfide bonds, multi-subunits assembly, electrostatic interactions, hydrophobic and hydrophilic interactions, or others, and any combination thereof. In some embodiments, properly processed refers to chemical posttranslational modification including, but not limited to, glycosylation (N′- and O′-), phosphorylation, acetylation, methylation, myristylation, palmitoylation, or others, and any combination thereof. In some embodiments, increased levels or properly processed peptide is at least 2-fold, 5-fold, 10-fold, 50-fold, 100-fold, or 1,000-fold more in a specific cell type compared to any other cell type, and any value and range therebetween. In some embodiments, increased levels or properly processed peptide is 2- to 5-fold, 4- to 10-fold, 8- to 50-fold, 40- to 150-fold, 100- to 500-fold, or 400- to 1,000-fold more in a specific cell type compared to any other cell type. Each possibility represents a separate embodiment of the invention. Methods of determining proper peptide processing are well known to one of skill in the art, and include non-limiting examples, such as, protease/peptidase assay, CD spectra, X-ray crystallography, NMR, fast parallel proteolysis, eastern blot, lectin chromatography, phosphorylation assay, and others.

According to some embodiments, the present invention is directed to delivery and expression of endogenous precursor proteins (i.e., propeptides). In one embodiment, a propeptide domain is encoded by a polynucleotide. In one embodiment, a propeptide domain is encoded by a chimeric polynucleotide of the invention. In some embodiments, translation of a chimeric polypeptide comprising a propeptide domain increases the translation efficiency of a modulating peptide. In some embodiments, after polypeptide of the present invention is in the secretory pathway, the propeptide domain is further cleaved off resulting in a mature peptide.

Non-limiting examples of specific cell predominantly expressed proteins are well known in the art and include: N′ and C′ procollagen in the muscle (PINP and PICP, respectively), factor X in the liver, insulin, glucagon, somatostatin and ghrelin in the pancreas, neuropeptides and neurotrophins in neurons, cytokines in immune cells, and others.

In some embodiments, the first polynucleotide of the invention encodes a propeptide comprising or consisting of the amino acid sequence:

(SEQ ID NO: 1)
MTILFLTMVISYFGCMKAAPMKEANIRGQGGLAYPGVRTHGTLESVNGPK
AGSRGLTSLADTFEHVIEELLDEDQKVRPNEENNKDADLYTSRVMLSSQV
PLEPPLLFLLEEYKNYLDAANIVISMIRVRR.

In some embodiments, the first polynucleotide encodes a propeptide comprising or consisting of the amino acid sequence:

(SEQ ID NO: 2)
MSMLFYTLITAFLIGIQAEPHSESNVPAGHTIPQAHWTKLQHSLDTALRR
ARSAPAAAIAARVAGQTRNITVDPRLFKKRRLRSPRVLFSTQPPREAADT
QDLDFEVGGAAPFNRTHRSKR.

In some embodiments, the first polynucleotide and the second polynucleotide are not derived from the same gene. In some embodiments, the first polynucleotide and the second polynucleotide are not derived from the same gene or genomic polynucleotide sequence.

In some embodiments, the first polynucleotide encoding the propeptide domain comprises a polynucleotide encoding the endogenous signal peptide and a polynucleotide encoding the endogenous protease/convertase motif.

In one embodiment, following expression and secretion, the signal peptide is cleaved from the precursor protein. In another embodiment, the term “protein” as used herein encompasses a “chimera polypeptide”.

As used herein, the term “protease/convertase” refers to a group of proteins capable of activating other inactivated target proteins by modifying the inactivated proteins, e.g., by means of proteolytic cleavage at a specific site or motif, thereby giving rise to the active isoform of their target proteins.

The terms “specific site”, “motif”, “cleavage site”, “proteolytic site” and “protease recognition site” are used herein interchangeably.

According to some embodiments, the present invention is directed to delivery and expression of a polynucleotide encoding a protease/convertase motif. In one embodiment, a protease/convertase motif is encoded by the first polynucleotide of the invention. In one embodiment, a protease/convertase motif is encoded by the chimeric polynucleotide of the invention. In one embodiment, a protease/convertase motif is an endogenous motif. In one embodiment, the protease/convertase motif is endogenous to the propeptide domain. In one embodiment, the protease/convertase motif endogenously proceeds the propeptide domain (e.g., located 3′ down-stream thereof). In one embodiment, the propeptide domain is removed from the mature peptide by cleavage at the protease/convertase motif. In one embodiment, a protease/convertase motif is recognized by a ubiquitous protease/convertase. In one embodiment, a protease/convertase motif is recognized by a cell specific protease/convertase. In one embodiment, a protease/convertase motif is recognized by a cell-specific protease/convertase. As used herein, the term “endogenous” and “endogenously” refers to that the propeptide domain, the signal peptide, and the protease/convertase polynucleotides are all parts of the same gene or product thereof, are transcribed from the same gene or located in the same genomic DNA region, or mRNA molecule transcribed therefrom, or protein translated therefrom.

In one embodiment, chimeric polynucleotide of the invention comprises a protease/convertase motif encoding polynucleotide ligated to a modulating peptide encoding polynucleotide.

In one embodiment, a protease/convertase motif is an exogenous motif. In one embodiment, chimeric polynucleotide of the invention comprises a protease/convertase motif encoding polynucleotide ligated to a propeptide domain encoding polynucleotide. In some embodiments, chimeric polynucleotide of the invention comprises a propeptide domain encoding polynucleotide ligated to a protease/convertase motif encoding polynucleotide ligated to a modulating peptide encoding polynucleotide.

In one embodiment, chimeric polypeptide of the invention comprises a protease/convertase motif in conjunction to a propeptide domain. In one embodiment, chimeric polypeptide of the invention comprises a protease/convertase motif in conjunction to a modulating peptide. In some embodiments, a chimeric polypeptide of the invention comprises a propeptide domain in conjunction to a protease/convertase motif in conjunction to a modulating peptide.

In some embodiments, the first polynucleotide encoding a propeptide does not comprise or consists of the endogenous mature peptide sequence resulting from the processing of the propeptide. In some embodiments, the second polypeptide encoding the modulating peptide does not comprise or consists of the mature polypeptide resulting from the processing of the propeptide encoded by the first polynucleotide. In some embodiments, the first and second polynucleotides are derived from different genes or from distinct genomic DNA regions.

In one embodiment, “complementary polynucleotide sequence” refers to a sequence, which results from reverse transcription of messenger RNA using a reverse transcriptase or any other RNA dependent DNA polymerase. In one embodiment, the sequence can be subsequently amplified in vivo or in vitro using a DNA polymerase.

In one embodiment, “genomic polynucleotide sequence” refers to a sequence derived (isolated) from a chromosome and thus it represents a contiguous portion of a chromosome.

In one embodiment, “chimeric polynucleotide sequence” refers to a sequence, which is at least partially complementary and at least partially genomic. In one embodiment, a chimeric sequence can include some exonal sequences required to encode the protein of the invention, as well as some intronic sequences interposing there between. In one embodiment, the intronic sequences can be of any source, including of other genes, and typically will include conserved splicing signal sequences. In one embodiment, intronic sequences include cis acting expression regulatory elements.

In another embodiment, the invention is directed to expression vectors comprising chimeric polynucleotides of the invention. In another embodiment, the invention further provides a cell comprising the expression vector of the invention. In another embodiment, a composition of the invention comprises the expression vector of the invention and/or a polynucleotide of the invention and/or a cell comprising the expression vector and a suitable carrier. As used herein, the terms “expression vector” and “delivery vector” are interchangeable.

In some embodiments, polynucleotides of the present invention are prepared using PCR techniques, or any other method or procedure known to one skilled in the art. In some embodiments, the procedure involves the legation of two different DNA sequences (See, for example, “Current Protocols in Molecular Biology”, eds. Ausubel et al., John Wiley & Sons, 1992).

In some embodiment, tissue-specific promoters suitable for use with the present invention include sequences which are functional in specific cell population, example include, but are not limited to promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). Inducible promoters suitable for use with the present invention include for example the tetracycline-inducible promoter (Srour, M. A., et al., 2003. Thromb. Haemost. 90: 398-405).

In one embodiment, polynucleotides of the present invention are inserted into expression vectors (i.e., a nucleic acid construct) to enable expression of the recombinant chimeric protein. In one embodiment, the expression vector of the present invention includes additional sequences which render this vector suitable for replication and integration in prokaryotes. In one embodiment, the expression vector of the present invention includes additional sequences which render this vector suitable for replication and integration in eukaryotes. In one embodiment, the expression vector of the present invention includes a shuttle vector which renders this vector suitable for replication and integration in both prokaryotes and eukaryotes. In some embodiments, cloning vectors comprise transcription and translation initiation sequences (e.g., promoters, enhances) and transcription and translation terminators (e.g., polyadenylation signals).

In one embodiment, a variety of prokaryotic or eukaryotic cells can be used as host-expression systems to propagate the polynucleotide of the present invention. In some embodiments, these include, but are not limited to, microorganisms, such as bacteria transformed with a recombinant bacteriophage DNA, plasmid DNA or cosmid DNA expression vector containing the protein coding sequence; yeast transformed with recombinant yeast expression vectors containing the protein coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the protein coding sequence.

In some embodiments, non-bacterial expression systems are used (e.g. mammalian expression systems such as CHO cells) to propagate the polynucleotides of the present invention in mammalian cells is pCI-DHFR vector comprising a CMV promoter and a neomycin resistance gene. Construction of the pCI-dhfr vector is described, according to one embodiment, in the examples section below.

As used herein, the term “propagate” refers to increase the copy number or number of polynucleotide molecules of the polynucleotide of the invention.

In some embodiments, in bacterial systems of the present invention, a number of expression vectors can be advantageously selected depending upon the use intended for the protein expressed. In one embodiment, large quantities of protein are desired. In one embodiment, vectors that direct the expression of high levels of the protein product, possibly as a fusion with a hydrophobic signal sequence, which directs the expressed product into the periplasm of the bacteria or the culture medium where the protein product is readily purified are desired. In one embodiment, certain fusion protein engineered with a specific cleavage site to aid in recovery of the protein. In one embodiment, vectors adaptable to such manipulation include, but are not limited to, the pET series of E. coli expression vectors [Studier et al., Methods in Enzymol. 185:60-89 (1990)].

In one embodiment, yeast expression systems are used. In one embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In one embodiment, the expression vector of the present invention further includes additional polynucleotide sequences that allow, for example, the translation of several proteins from a single mRNA such as an internal ribosome entry site (IRES) and sequences for genomic integration of the promoter-chimeric protein.

In some embodiments, mammalian expression vectors include, but are not limited to, pcDNA3, pcDNA3.1(+/−), pGL3, pZeoSV2(+/−), pSecTag2, pDi splay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the proteins of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

In one embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

In one embodiment, in vivo gene therapy using EPO has been attempted in animal models such as rodents [Bohl et al., Blood. 2000; 95:2793-2798], primates [Gao et al., Blood, 2004, Volume 103, Number 9] and has proven successful in human clinical trials for patients with chronic renal failure [Lippin et al Blood 2005, 106, Number 7].

In one embodiment, plant expression vectors are used. In one embodiment, the expression of a protein coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the protein), expression constructs of the present invention can also include sequences engineered to optimize stability, production, purification, yield or activity of the expressed protein, all of which are well known to a skilled artisan.

Various methods, in some embodiments, can be used to introduce the expression vector of the present invention into the host cell system. In some embodiments, such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, transformed cells are cultured under effective conditions, which allow for the expression of high amounts of recombinant protein. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant protein of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, the terms “polypeptide” or “protein” as used herein encompasses native polypeptides or peptides (either degradation products, synthetically synthesized polypeptides, proteins, chimeras, or recombinant polypeptides) and peptidomimetics (typically, synthetically synthesized proteins), as well as peptoids and semipeptoids which are protein analogs, which have, in some embodiments, modifications rendering the protein of the invention more stable while in a body or more capable of penetrating into tissues and cells.

The term “linker” refers a molecule or macromolecule serving to connect different moieties of a peptide or a polypeptide. In one embodiment, said linker may also facilitated other functions, including, but not limited to, preserving biological activity, maintaining sub-units and domains interactions, and others.

In some embodiments, the chimeric polypeptides of the invention comprise linkers. In another embodiment, the linker may be biodegradable such that the chimeric polypeptide of the invention is further processed by hydrolysis and/or enzymatic cleavage inside cells. In one embodiment, cell specifically-expressed proteases, can be used in the delivery of prodrugs of cytotoxic agents, with the linker being selective for a site-specific proteolysis. In some embodiments, readily-cleavable groups include acetyl, trimethylacetyl, butanoyl, methyl succinoyl, t-butyl succinoyl, ethoxycarbonyl, methoxycarbonyl, benzoyl, 3-aminocyclohexylidenyl, and the like.

In one embodiment, a polypeptide of the invention is synthesized using a single polynucleotide encoding the polypeptide of the invention. In some embodiments, the polynucleotide encoding the polypeptide of the invention is ligated into an expression vector, comprising a transcriptional control of a cis-regulatory sequence (e.g., promoter sequence). In some embodiments, the cis-regulatory sequence is suitable for directing constitutive expression of the polypeptide of the invention. In some embodiments, the cis-regulatory sequence is suitable for directing tissue specific expression of the polypeptide of the invention. In some embodiments, the cis-regulatory sequence is suitable for directing inducible expression the polypeptide of the invention.

In some embodiments of the methods described herein, inhibiting is reducing by more than 2%, reducing by more than 5%, reducing by more than 10%, reducing by more than 25%, reducing by more than 50%, reducing by more than 75%, reducing by more than 90%, reducing by more than 95%, or reducing by more than 99%, and any value and range therebetween. In some embodiments of the methods described herein, inhibiting is reducing by 2-10%, reducing by 5-15%, reducing by 10-25%, reducing by 20-50%, reducing by 45-70%, reducing by 65-85%, reducing by 80-95%, or reducing by 90-100%. Each possibility represents a separate embodiment of the invention.

The terms “inhibiting”, “blocking”, “reducing” and “attenuating” are interchangeable.

In some embodiments of the methods described herein, increasing is elevating by more than 2%, elevating by more than 5%, elevating by more than 25%, elevating by more than 50%, elevating by more than 100%, elevating by more than 150%, elevating by more than 250%, elevating by more than 500%, or elevating by 1,000%, and any value and range therebetween. In some embodiments of the methods described herein, increasing is elevating by 2-15%, elevating by 5-25%, elevating by 20-75%, elevating by 50-150%, elevating by 100-250%, elevating by 200-550%, elevating by 500-750%, or elevating by 750-1,000%. Each possibility represents a separate embodiment of the invention.

In some embodiments of the methods described herein, increasing is elevating by more than 2-fold. In some embodiments of the methods described herein, increasing is elevating by more than 5-fold. In some embodiments of the methods described herein, increasing is elevating by more than 10-fold. In some embodiments of the methods described herein, increasing is elevating by more than 20-fold. In some embodiments of the methods described herein, increasing is elevating by more than 50-fold. In some embodiments of the methods described herein, increasing is elevating by more than 100-fold.

The terms “increasing”, “elevating” and “amplifying” are interchangeable.

### Pharmaceutical Compositions

According to some embodiments, the invention is directed to a pharmaceutical composition comprising as an active ingredient a therapeutically effective amount of the chimeric polynucleotide of the invention or delivery vector comprising thereof, and pharmaceutically acceptable carrier and/or diluents. In some embodiments, the pharmaceutical composition facilitates administration of a compound to an organism.

According to some embodiments, a cell comprising the chimeric polynucleotide of the invention, or a delivery vector comprising thereof, is provided.

According to some embodiments, a composition comprising a cell comprising the chimeric polynucleotide of the invention, or a delivery vector comprising thereof, is provided. In some embodiments, a composition comprising the cell comprising the chimeric polynucleotide of the invention, or a delivery vector comprising thereof, and a carrier, is provided.

In some embodiments, the cell is selected from: sensory neurons, motor neurons, interneurons, neurons of the brain, astrocytes, microglia, ependymal cells, oligodendrocytes, Schwann cells, satellite cells, enteric glial cells, olfactory cells, and sheathing cells. In some embodiments, the cell is selected from: red skeletal muscle cell, white skeletal muscle cell, intermediate muscle cell, nuclear bag cell, nuclear chain cell, satellite cell, heart muscle cell, nodal heart muscle cell, Purkinje fiber cell, smooth muscle cell, and myoepithelial cell. In some embodiments, the cell is selected from: hepatocyte, Kupffer cell, and liver stellate cell. In some embodiments, the cell is selected from: Alpha cell, Beta cell, Delta cell, Gamma cell, and Epsilon cell.

As used herein, the term “carrier” refers to a diluent, adjuvant, excipient, or vehicle with which the therapeutic compound is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from about 0.1% to about 99.99999% by weight of the pharmaceutical compositions presented herein.

As used herein, the term “pharmaceutically acceptable” means suitable for administration to a subject, e.g., a human. For example, the term “pharmaceutically acceptable” can mean approved by a regulatory agency of the Federal or a state government or listed in the U. S. Pharmacopeia or other generally recognized pharmacopeia for use in animals, and more particularly in humans.

An embodiment of the invention relates to polynucleotides of the present invention, presented in unit dosage form and prepared by any of the methods well known in the art of pharmacy. In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

According to one embodiment, the compositions of the present invention are administered in the form of a pharmaceutical composition comprising at least one of the active components of this invention (e.g., the chimeric polynucleotide or a delivery vector comprising thereof) together with a pharmaceutically acceptable carrier or diluent. In another embodiment, the compositions of this invention can be administered either individually or together in any conventional oral, parenteral or transdermal dosage form. In some embodiments, the pharmaceutical composition further comprises at least one anticancer agent such as a chemotherapeutic agent. In some embodiments, the pharmaceutical composition is adopted for combined administration with an anticancer therapy such as chemotherapy, radiotherapy, immunotherapy, hormonal therapy, toxin therapy or surgery.

As used herein, the terms “administering”, “administration”, and like terms refer to any method which, in sound medical practice, delivers a composition containing an active agent to a subject in such a manner as to provide a therapeutic effect.

Depending on the location of the tissue of interest, the chimeric polynucleotide or delivery vector of the present invention can be administered in any manner suitable for the provision of the mentioned herein molecules to cells within the tissue of interest. Thus, for example, a composition containing the chimeric polynucleotide or delivery vector of the present invention can be introduced, for example, into the systemic circulation, which will distribute the peptide to the tissue of interest. Alternatively, a composition can be applied topically to the tissue of interest (e.g., injected, or pumped as a continuous infusion, or as a bolus within a tissue, applied to all or a portion of the surface of the skin, etc.).

In some embodiments, the pharmaceutical compositions comprising the chimeric polynucleotide or delivery vector of the current invention are administered via oral, rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical compositions of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

For topical application, the chimeric polynucleotide or delivery vector of the present invention, derivative, analog or a fragment thereof can be combined with a pharmaceutically acceptable carrier so that an effective dosage is delivered, based on the desired activity. The carrier can be in the form of, for example, and not by way of limitation, an ointment, cream, gel, paste, foam, aerosol, suppository, pad or gelled stick.

For oral applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. The tablets of the invention can further be film coated.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

The presently described chimeric polynucleotide or delivery vector, derivatives, or analogs thereof may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The compositions also include incorporation of the active material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In one embodiment, it will be appreciated that the chimeric polynucleotide or delivery vector of the present invention can be provided to the individual with additional active agents to achieve an improved therapeutic effect as compared to treatment with each agent by itself. In another embodiment, measures (e.g., dosing and selection of the complementary agent) are taken to adverse side effects which are associated with combination therapies.

In one embodiment, depending on the severity and responsiveness of the condition to be treated, dosing can be of a single or a plurality of administrations, with course of treatment lasting from several days to several weeks or until cure is affected or diminution of the disease state is achieved.

In some embodiments, the chimeric polynucleotide or delivery vector are administered in a therapeutically safe and effective amount. As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the presently described manner. In another embodiment, a therapeutically effective amount of the chimeric polynucleotide or delivery vector is the amount of the mentioned herein molecules necessary for the in vivo measurable expected biological effect. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21^{st} Ed., Lippincott Williams & Wilkins, Philadelphia, Pa., (2005). In some embodiments, preparation of effective amount or dose can be estimated initially from in vitro assays. In one embodiment, a dose can be formulated in animal models and such information can be used to more accurately determine useful doses in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

Pharmaceutical compositions containing the presently described chimeric polynucleotide or delivery vector as the active ingredient can be prepared according to conventional pharmaceutical compounding techniques. See, for example, Remington's Pharmaceutical Sciences, 18^{th} Ed., Mack Publishing Co., Easton, Pa. (1990). See also, Remington: The Science and Practice of Pharmacy, 21^{st} Ed., Lippincott Williams & Wilkins, Philadelphia, Pa. (2005).

In one embodiment, compositions of the present invention formulated with a compatible pharmaceutical carrier are prepared, placed in an appropriate container, and labeled for treatment of an indicated condition.

In one embodiment, compositions of the present invention are presented in a pack or dispenser device, such as an FDA approved kit, which contains, one or more unit dosages forms containing the active ingredient. In one embodiment, the pack, for example, comprises metal or plastic foil, such as a blister pack. In one embodiment, the pack or dispenser device is accompanied by instructions for administration. In one embodiment, the pack or dispenser is accommodated by a notice associated with the container in a form prescribed by a governmental agency regulating the manufacture, use or sale of pharmaceuticals, which notice is reflective of approval by the agency of the form of the compositions or human or veterinary administration. Such notice, in one embodiment, is labeling approved by the U.S. Food and Drug Administration for prescription drugs or of an approved product insert.

### Treatment and Use of Compositions

According to some embodiments, a method for treating, ameliorating, reducing and/or preventing a condition related to cell-specific-associated disease or disorder in a subject in need thereof, comprising the step of: administering to a subject a pharmaceutical composition comprising an effective amount of the chimeric polynucleotide of the invention, thereby treating, ameliorating, reducing and/or preventing a condition related to cell-specific-associated disease or disorder in the subject, is provided.

According to some embodiments, a method for treating or ameliorating a cell-specific-associated disease or disorder in a subject in need thereof, comprising administering to the subject any one of:

(i) a chimeric polynucleotide of the invention;
(ii) a delivery vector of the invention; or
(iii) a pharmaceutical composition of the invention,
thereby treating or ameliorating cell-specific-associated disease or disorder in the subject, is provided.

In some embodiments, the method of the invention comprises contacting a cell selected from: sensory neurons, motor neurons, interneurons, neurons of the brain, astrocytes, microglia, ependymal cells, oligodendrocytes, Schwann cells, satellite cells, enteric glial cells, olfactory cells, and sheathing cells, with the chimeric polynucleotide of the invention or a delivery vector comprising thereof.

In some embodiments, the method of the invention comprises contacting a cell selected from: red skeletal muscle cell, white skeletal muscle cell, intermediate muscle cell, nuclear bag cell, nuclear chain cell, satellite cell, heart muscle cell, nodal heart muscle cell, Purkinje fiber cell, smooth muscle cell, and myoepithelial cell, with the chimeric polynucleotide of the invention or a delivery vector comprising thereof.

In some embodiments, the method of the invention comprises contacting a cell selected from: hepatocyte, Kupffer cell, and liver stellate cell, with the chimeric polynucleotide of the invention or a delivery vector comprising thereof.

In some embodiments, the method of the invention comprises contacting a cell selected from: Alpha cell, Beta cell, Delta cell, Gamma cell (i.e., PP cell), and Epsilon cell, with the chimeric polynucleotide of the invention or a delivery vector comprising thereof.

In some embodiments, the use of a composition comprising an effective amount of the chimeric polynucleotide or delivery vector comprising thereof in the preparation of a medicament for the treatment, amelioration, reduction, or prevention of a cell-specific-associated disease or disorder in a subject in need thereof, is provided. In some embodiments, the invention is directed to the use of a composition comprising an effective amount of one or more the chimeric polynucleotide or delivery vector comprising thereof in the preparation of a medicament for the treatment of a cell-specific-associated or disorder in a subject in need thereof.

In one embodiment, the chimeric polynucleotide of the present invention is provided to the subject per se. In one embodiment, one or more of the chimeric polynucleotides of the present invention are provided to the subject per se. In one embodiment, the chimeric polynucleotide of the present invention is provided to the subject as part of a pharmaceutical composition where it is mixed with a pharmaceutically acceptable carrier. In one embodiment, one or more of the chimeric polynucleotides of the present invention are provided to the subject as part of a pharmaceutical composition where they are mixed with a pharmaceutically acceptable carrier.

In one embodiment, the vector of the present invention is provided to the subject per se. In one embodiment, one or more of the vectors of the present invention are provided to the subject per se. In one embodiment, the vector of the present invention is provided to the subject as part of a pharmaceutical composition where it is mixed with a pharmaceutically acceptable carrier. In one embodiment, one or more of the vectors of the present invention are provided to the subject as part of a pharmaceutical composition where they are mixed with a pharmaceutically acceptable carrier.

As used herein, the term “a disease or disorder” includes but not limited to biological, chemical, physical, electrical, or structural abnormalities in components of a tissue, in a way which deviates from standard homeostasis.

As used herein “developmental disease or disorder” is a physiologically based condition that appears early in childhood, typically before school entry.

As used herein “cancer disease” is a disease associated with cell proliferation.

The term “subject” as used herein refers to an animal, more particularly to non-human mammals and human organism. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig, and pig. In one embodiment, the subject is a human. Human subjects may also include fetuses. In one embodiment, a subject in need thereof is a subject afflicted with and/or at risk of being afflicted with a condition associated with neural disease or disorder. In one embodiment, a subject in need thereof is a subject afflicted with and/or at risk of being afflicted with a condition associated with increased neural cell proliferation.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described peptides prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

As used herein, the term “condition” includes anatomic and physiological deviations from the normal that constitute an impairment of the normal state of the living animal or one of its parts, that interrupts or modifies the performance of the bodily functions.

Any concentration ranges, percentage range, or ratio range recited herein are to be understood to include concentrations, percentages or ratios of any integer within that range and fractions thereof, such as one tenth and one hundredth of an integer, unless otherwise indicated.

Any number range recited herein relating to any physical feature, such as polymer subunits, size or thickness, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds.), “Basic and Clinical Immunology” (8th Edition), Appleton & Lange, Norwalk, Conn. (1994); Mishell and Shiigi (eds.), “Strategies for Protein Purification and Characterization—A Laboratory Course Manual” CSHL Press (1996); all of which are incorporated by reference. Other general references are provided throughout this document.

### Materials and Methods

### Cell Lines

293FT—The inventors utilized Human Embryonic Kidney 293 (HEK293) cells for production of lentiviral vectors expressing BDNF or small inhibitory peptides (propeptide; as described hereinbelow).

COS-7 and 3T3/NIH—The inventors used 3T3/NIH and COS-7 cells for viral transduction (as described hereinbelow) with BDNF and propeptide expressing viruses to obtain proper BDNF biogenesis.

### Cell Maintenance

The complete growth medium used by the inventors contained: Dulbecco's Modified Eagle's Medium, fetal bovine serum (final concentration of 10%), penicillin-streptomycin antibiotics and L-glutamine. Routine maintenance of the culture was performed as follows: cells were grown in 100 mm plates until reaching confluence of 80-90%, passaged every 2-3 days and incubated in humidified incubator set for 37° C. with an atmosphere containing 5% CO₂.

### SDS-PAGE and Western Blotting

Lysis of cells was performed using sample buffer x2 plus BME (1:9), in order to extract the intracellular proteins. The process was performed on ice, to reduce proteins' degradation. Each lane of a polyacrylamide gel was loaded with the same amount of lysate and subjected to electrophoresis (PAGE) in the presence of sodium dodecyl sulfate (SDS). Proteins were separated on 10% or 15% polyacrylamide gels and transferred to nitrocellulose membranes. The membranes were blocked with 5% BSA in TBS-T and probed with one of the following primary antibodies diluted in blocking solution: anti β-Actin (1:12,000), anti IκB (1:2,000), anti BDNF (1:1,000), anti proBDNF (1:500) or anti HA-tag (1:200), at 4° C. overnight. One the second day, the inventors washed the membranes three times with TBS-T, after which they were incubated with one of the following secondary antibodies diluted in TBS-T solution: anti-Mouse HRP conjugated, anti-Rabbit HRP conjugated or anti-Goat HRP conjugated (all at 1:10,000), at room temperature for 1 hr. Thereafter, the inventors 3 washing steps with TBS-T were employed, membranes were visualized with ECL kit (Thermo) and bands were quantified by Quantity-one software. Each sample was normalized to β-Actin levels and quantified relatively to control samples.

### Molecular Cloning

Molecular cloning was performed in order to transfer small inhibitory peptide construct into a viral vector carrier. The open reading frame (ORF) encoding a small inhibitory peptide was cloned into a viral plasmid using the AgeI and XhoI restriction enzymes. The DNA segment was joined by the enzyme T4 DNA ligase, after which the inventors transformed the ligated vector into DH5α strain of E. coli cell by electroporation. Following bacterial cells transformation, colonies were selected on Amp-R LB-agar plates. Plasmid DNA isolation from was performed using the Miniprep GenElute™ Plasmid kit (QIAGEN) and analyzed using restriction followed by electrophoresis on 1% (w/v) agarose gel containing ethidium bromide (final concentration of approximately 0.2 μg/ml). The gel electrophoresis was performed under 120 V, until the dye front reached 75-80% of the gel's length. The gel was then visualized under UV light, and DNA fragments were excised, recovered and concentrated using the Gel/PCR DNA Fragments Extraction Kit (GE Healthcare). For large-scale DNA plasmid isolation from the E. coli cultures, the inventors used a Maxiprep Plasmid Kit (QIAGEN).

### Cell Transfection

Transfection of Poly(I:C) into NIH3T3 was done using the PolyJet reagent according to manufacturer's instructions. Briefly, cells were cultured in a 6 well plate. On the day of transfection, the medium was replaced by a fresh one. Transfection mix was prepared by adding either 0.25 or 1 μg Poly(I:C) and then 200 μl PolyJet to DMEM medium. The mix was incubated for 10 min at RT and added to the cells. The cells were incubated for 24 hr at 37° C. with 5% CO₂.

### Viral Vectors Production

High titter preparation of lentivirus (LV) stocks was performed by the transient co-transfection of 293FT packaging cells. First, cells were cultured on Poly-L-Lysine (PLL) covered plates until they reach 80-90% confluence. Co-transfection was performed in serum-free DMEM medium, mediated by PEI reagent. DNA mix was prepared, using the following plasmids:

Transfer vector (12 μg): cloned with gene of interest.

pMDL (7.8 μg): gag elements and pol elements.

pRSV-Rev (3 μg): encoding reverse transcriptase enzyme.

VSV-G (4.2 μg): envelope elements.

On the next day the medium was replaced with a fresh 10% hiFBS DMEM medium and the cells were further incubated for 24 hr. The medium was collected, filtered and subjected to centrifugation of 19,000 rpm for 2.5 hr at 15° C. The supernatant was decanted, and viral pellet was resuspended in 50 μl HBSS, aliquoted and stored in −80° C.

### Viral Transduction of Cell Lines

Relevant cells (3T3/NIH, COS-7) were seeded in a 24 wells plate (2.5×10⁴ cells/well), in 250 μl medium containing 8 μg/ml Polybrene. Cells were infected with 10 μl of virus for a period of 24 hr. Next, the cells were supplemented with fresh medium (250 μl) and incubated for an additional 24 hr. The green fluorescent protein (GFP) was visualized under fluorescent microscope and the transduced cells were further analyzed according to relevant procedures.

### Immunofluorescence (IF)

Relevant cells (COS-7, 3T3/NIH) were seeded in a 12 wells plate containing glass cover slips coated with PLL (1:10) and were grown to 50-70% confluence. Following viral infection, the cells were fixed in 4% paraformaldehyde (in PBS) for 15 min and permeabilized with 0.1-0.3% Triton X-100 (in PBS) for 10 min. Blocking was performed in a saturation buffer for 1 hr at room temperature. Following these treatments, cells were incubated overnight at 4° C. with one of the following primary antibodies diluted in saturation buffer: anti HA-tag or anti proBDNF. On the next day the cells were washed three times with PBS and incubated for 1 hr at room temperature with the secondary antibody, Alexa Fluor-488 donkey α-rabbit IgG, diluted to a final concentration of 1:500 in saturation buffer, after which cells were washed three times with PBS. Nuclei were stained with 4′, 6-Diamidino-2-phenylindole dihydrochloride (DAPI) and mounting was done with mounting medium. Images were taken by Olympus IX81 microscope with Hamamatsu ORCA R2 camera controlled by cellSens Dimension software. The same exposure conditions were used for all slides and images.

### Mice

Adult C57BL/6 male mice (age 5-10 weeks, weighing 18-25 gr) were used for virus injections and behavioral procedures. Animals were housed individually and maintained on a 12-hr light/dark cycle prior and subsequent to virus injection procedures. All procedures were performed in strict accordance with the University of Haifa regulations and the US National Institutes of Health guidelines.

### Microinjection of Viral Vectors

Mice were restrained in a stereotactic apparatus (KOPF, USA), anesthetized with 4% Isoflurane (USP 100%, Terrell) which was reduced to 1.5% throughout the surgery. Microinjection of the virus was performed by directly injecting 1 μl (0.1 μl/min) to the hippocampal CM region (AP: −1.78, LM: ±1.20, DV: −1.63, relative to Bregma). The injection was performed using a 10 μl micro syringe (#7653-01, HAMILTON). Following the surgery, animals were treated with an analgesic (dipyrone 50%, i.m.) and topical antibiotic (Synthomycine 5%), and allowed to recuperate for at least a week before the recording.

### Morris Water Maze

Mice were transferred to the Morris water maze (MWM) training room 5 min prior to the test. The learning procedure was performed in four daily trials for 5 days at 1 min per trial. In this learning experiment, animals learned to use cues to navigate to a hidden platform when placed at different, random locations around the perimeter of black circular tank (120 cm; 50 cm) filled with opaque water made with white nontoxic paint. The tank was divided into 4 quadrants, one of which contained a hidden platform during the entire learning process. During the learning process, escape latency to the hidden platform was measured in each trial and the learning curve was calculated for all training days. To assess reference memory after learning, a probe trial was performed 24 hr following the 3^{rd} day, in which the platform was removed from the tank. Percentage of time spent in each quadrant along the entire probe test was measured, and the data were analyzed by EthoVision XT 9 software (Noldus Information Technology, Canada).

### Example 1

### Design of a LV Construct for Viral-Mediated Small Inhibitory Peptide Expression

As small peptides suffer from low bioavailability, the inventors have utilized the lentiviral (LV) expression system in order to both deliver and endogenously express small peptides. It is well established that peptides are continuously produced by cells from large precursors that undergo specified biogenesis, the inventors thus chose a strategy by which LV mediated expression of a specific, large and natural precursor will facilitate artificial small peptide expression while exploiting the endogenous biogenesis machinery. Accordingly, the inventors designed a transgene expressing the large BDNF precursor which includes the pre and pro-BDNF domains. The mature BDNF sequence was replaced with a peptide construct which includes a cell penetrating motif, a HA-tag for detection, and a peptide sequence (FIG. 1; ‘TAT-HA-PEP’). The inventors' hypothesis was that the generated transgene will undergo proteolytic cleavage mimicking BDNF biogenesis, which will result in small peptide expression. As a control, the inventors designed a vector expressing the native BDNF.

### Example 2

### Viral Infection of COS-7 and 3T3/NIH Cell Lines Increased BDNF Expression

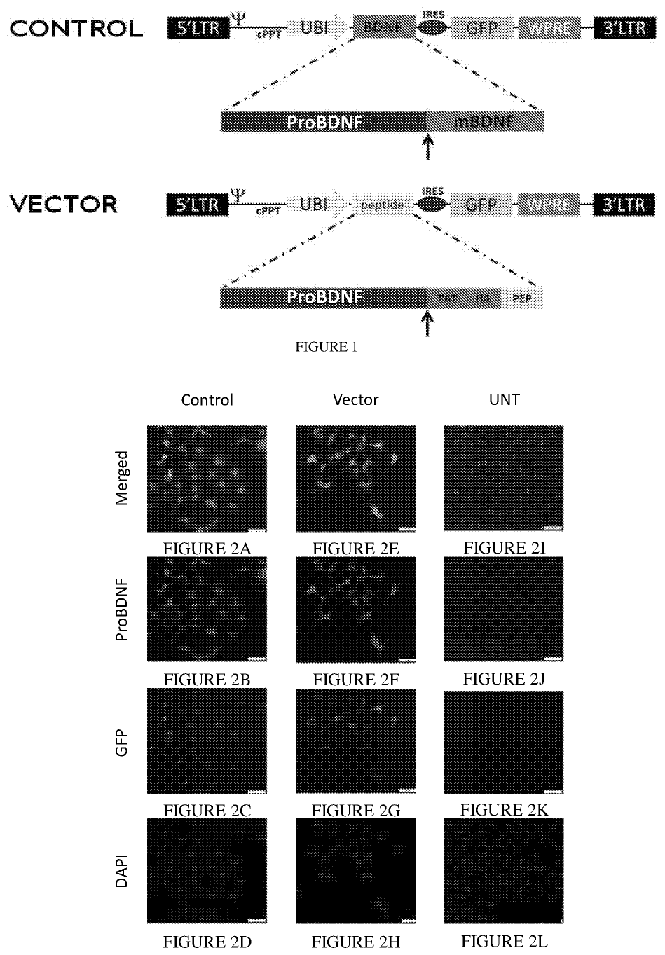

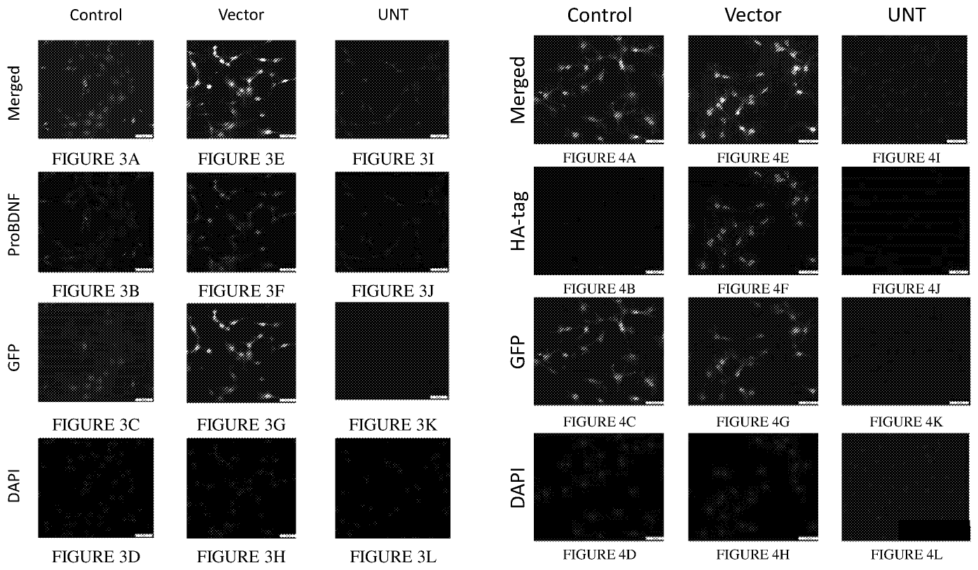

In order to test the above described system, the inventors selected COS-7 (African green monkey kidney fibroblast-like cell line) and 3T3/NIH (Mouse embryo fibroblast) cell lines, as both are known in the literature to endogenously express, properly produce, and secrete active mature BDNF. As a preliminary validation, the inventors examined endogenous BDNF expression, of both BDNF and the propeptide ‘TAT-HA-PEP’, following viral infection. To do so, the inventors used an immunofluorescent assay using a specific antibody targeted at the proBDNF domain. Since the proBDNF sequence was present in both viral vectors, the inventors decided to use the anti proBDNF antibody rather than an anti BDNF antibody. Uninfected COS-7 (FIG. 2) and 3T3/NIH (FIG. 3) cells were shown to express BDNF precursor endogenously, however, the expression is further increased following the mentioned above viral infection.

### Example 3

### Infected COS-7 and 3T3/NIH Cells Expressed a Peptide Construct

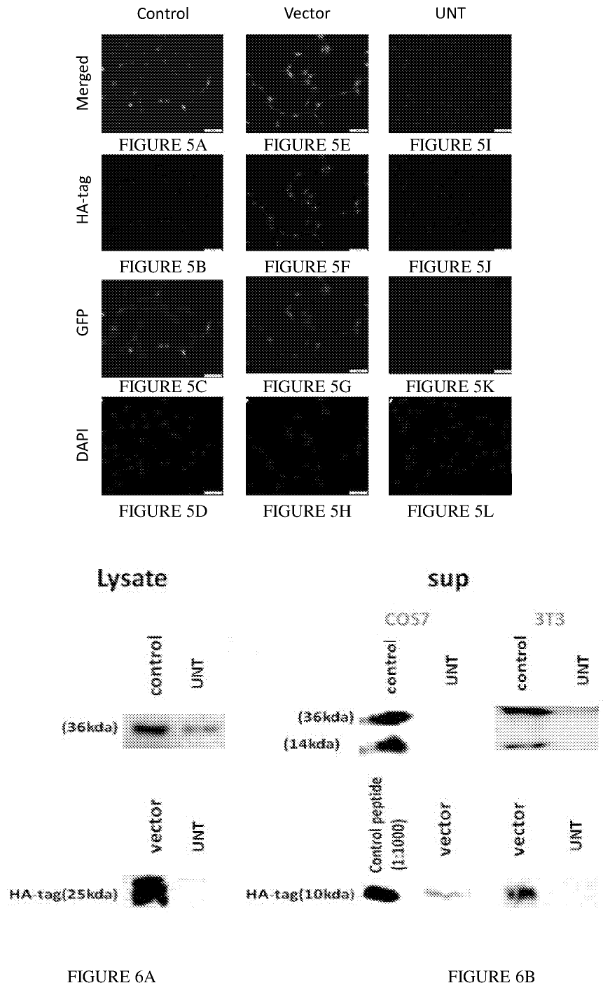

Next, the inventors determined and validated whether the peptide precursor expressed in infected COS-7 and 3T3/NIH cells was the ‘TAT-HA-PEP’ propeptide. Accordingly, the inventors used an HA-tag antibody. Indeed, the presence of the peptide precursor was only detected in the propeptide LV infected COS-7 (FIG. 4) and 3T3/NIH (FIG. 5) cells. Nonetheless, whether the artificial peptide precursor undergoes the relevant proteolytic cleavage and biogenesis, which will result in small peptide production within the cells, was yet to be determined.

### Example 4

### A propeptide transgene undergoes BDNF-like processing

Next, the inventors examined whether the peptide precursor undergone cleavage to produce the small peptide. To do so, COS-7 and 3T3/NIH cells were infected with the BDNF or ‘TAT-HA-PEP’ LVs (also termed BPEP), cell were lysed, and lysates were subsequently analyzed by western-bolt. As shown (FIG. 6A), in BDNF-infected cells, which over expressed the BDNF construct, the inventors had detected the proBDNF precursor (36 kDa) but not the cleaved mature BDNF (upper panel). Similarly, in the BPEP-infected cells, the inventors detected the un-cleaved form of the peptide precursor (25 kDa; lower panel). These results led the inventors to the assumption that most of the processed peptide, as well as the mature BDNF, might be secreted from the cells to the medium. To examine this hypothesis, the inventors collected the media from BDNF- and BPEP-infected cell cultures. The media were lyophilized (concentrated by about by 5-fold), and subsequently assayed by western-blot analysis. As shown (FIG. 6B), in medium of BDNF-infected cells, both the un-cleaved precursor and the cleaved mature BDNF were detected (36 kDa and 14 kDa, respectively; upper panel). Similarly, in medium of BPEP-infected cells, the cleaved small peptide (10 kDa) was detected (lower panel; a synthetic peptide comprising the same amino acid sequence was used a size marker). Thus, the results indicated that 3T3/NIH cells expressed and properly produced active mature BDNF, and the 3T3/NIH BPEP-infected cells, expressed the cloned construct and its mRNA was translated into the corresponding protein.

### Example 5

### Virally-Mediated Peptide 1 Expression Results in Decreased IκB Degradation in Poly I:C Transfected Cells

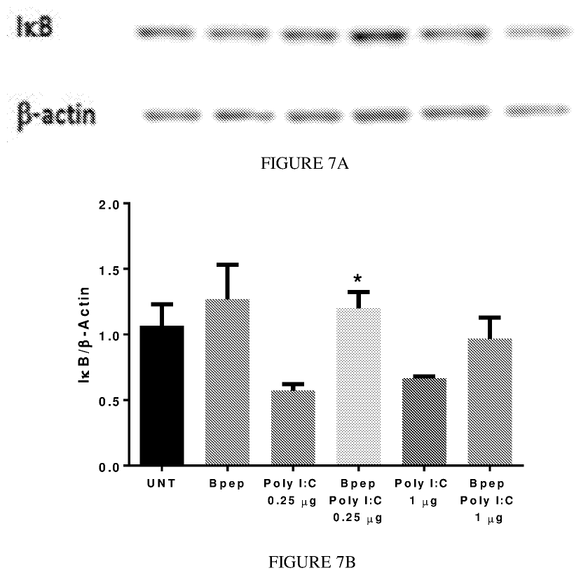

The above described data (i.e., example 4) indicated that the propeptide precursor undergone proper biogenesis and was secreted to the cell medium, similar to both endogenous and recombinant BDNF. Accordingly, the inventors further tested the functionality of the viral mediated peptide in vitro. As shown (FIG. 7)Error! Reference source not found., Poly I:C transfection resulted in decreased IκB levels. On the contrary, propeptide infected cells exhibited marginally decreased degradation of IκB following either 0.25 μg or 1 μg of Poly I:C transfection. (Univariate ANOVA: F (5, 13)=2.089, p<0.05. post hoc analysis, LSD test: Poly I:C 0.25 μg vs BPEP: p=0.029, Poly I:C 1 μg vs BPEP: p=0.031 Poly I:C 0.25 μg vs Poly I:C+Bpep: p=0.057).

### Example 6

### Vector-Mediated Peptide Delivery into CA1 Enhances Spatial Learning and Memory

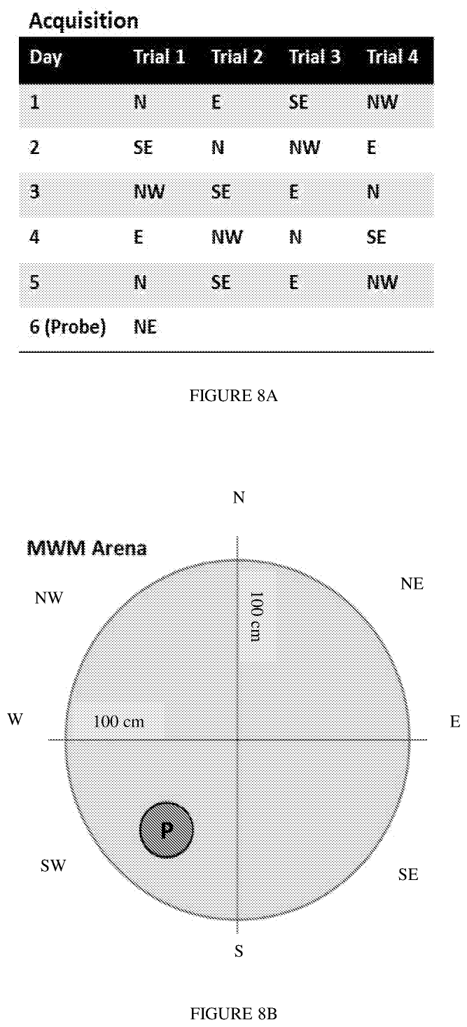

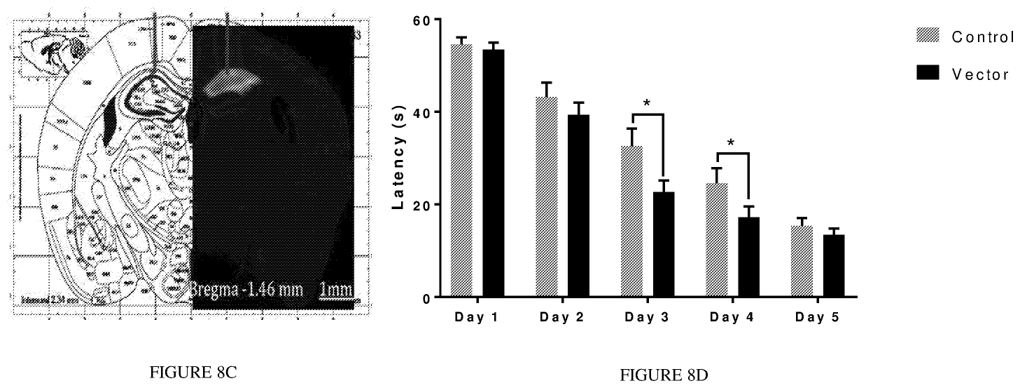

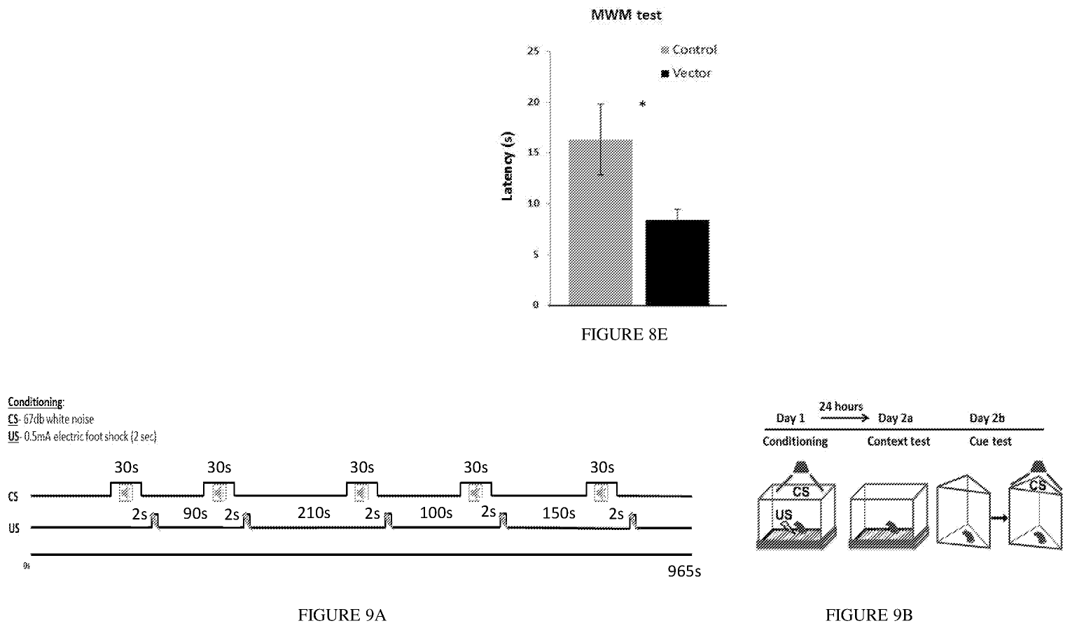

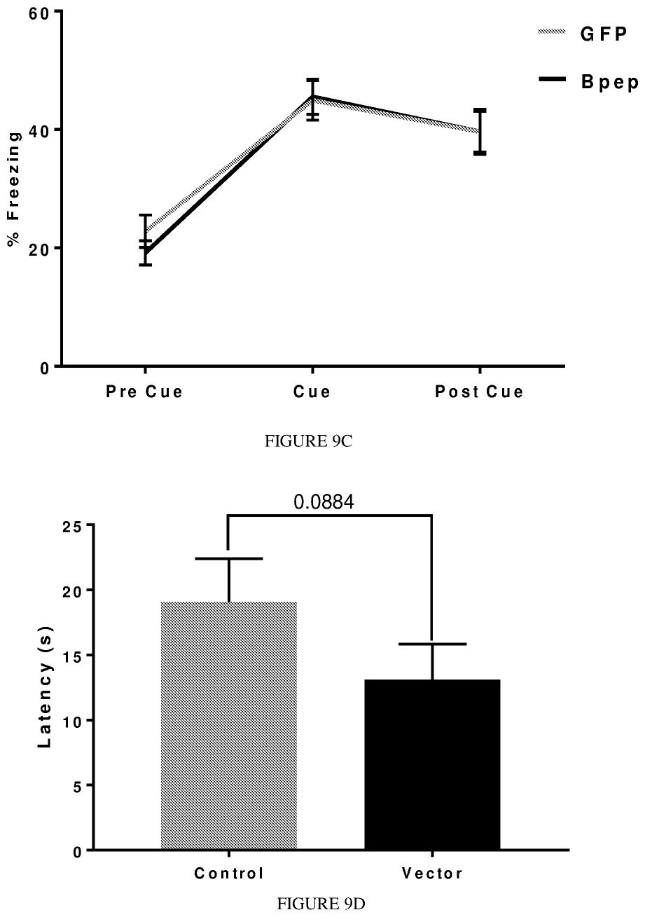

To further examine the function of the viral mediated peptide in vivo, the inventors injected the LV vector (comprising either the BPEP propeptide, or GFP as control) into the CA1 region of the hippocampus of naïve animals. First, spatial memory was compared between propeptide injected mice and GFP injected animals (Error! Reference source not found. FIG. 8) using MWM. Propeptide-infected mice were slightly more successful than the control mice in learning the position of hidden platform (FIG. 8D). Following the learning period of the MWM task, probe tests were performed 24 h after the 3rd day of training. Similarly, propeptide-injected mice exhibited a tendency for shorter escape latency to the target that previously contained the hidden platform, indicating that propeptide-injected mice had an enhanced spatial memory compared to the control group (FIG. 8E; t (7)=−1.935, p=0.08). Due to the MWM results, the inventors further examined the functionality of the viral mediated peptide in additional behavioral paradigm. The inventors compared contextual fear memory between propeptide injected mice and GFP injected animals (FIG. 9). Mice were tested for context fear conditioning 24 hr after conditioning, in the original training chamber, and freezing was used as the parameter of conditional fear. The results revealed significant difference in freezing percentage in propeptide treated animals compared to GFP 24 h post conditioning.

### Example 7

### Vector Mediated EPE Expression Results in Inhibition of ERK1/2 Nuclear Translocation

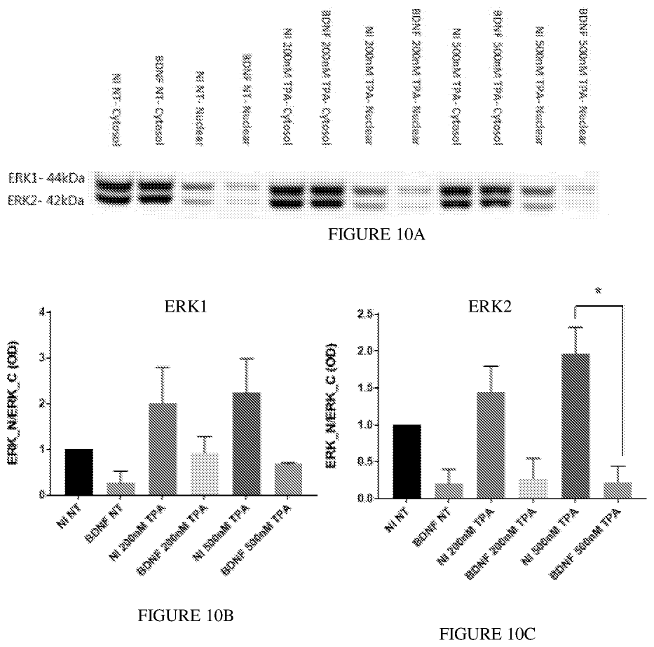

The inventors further examined whether infection with LV expressing the proBDNF, in which the mature BDNF coding sequence has been replaced with the sequence of NTS-derived phosphomimetic peptide (EPE peptide), would inhibit ERK translocation in COS7 cell line as well. To test that, COS7 cells were infected with the viral vector or left un-treated. Following infection, cells were stimulated with TPA (200 nM or 500 nM) for 15 min. ERK levels were tested in the nucleus and cytosol fractions by western-blot analysis. The results suggest that viral mediated expression of EPE peptide using the proBDNF precursor inhibited ERK nuclear translocation (FIG. 10).

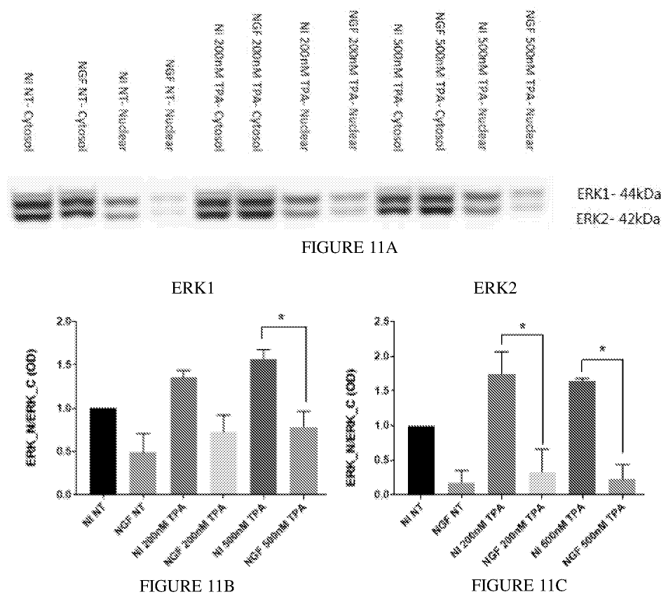

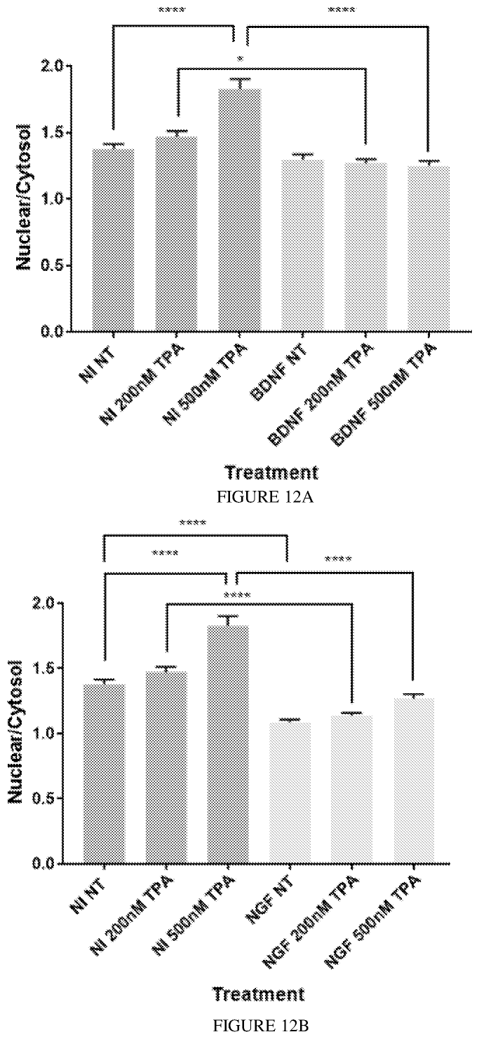

The inventors then tested whether a different precursor is applicable according to the disclosed invention. proBDNF was replaced by proNGF. COS7 cells were infected with LV expressing the proNGF, in which the mature NGF coding sequence has been replaced with the sequence of NTS-derived phosphomimetic peptide (EPE peptide). Following infection, cells were stimulated with TPA (200 nM or 500 nM) for 15 min. ERK levels were tested in the nucleus and cytosol fractions by western-blot analysis (FIG. 11). The results suggest that viral mediated expression of EPE peptide using the proNGF precursor inhibited ERK nuclear translocation. The results were also confirmed using immunofluorescence (FIG. 12).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A chimeric polynucleotide molecule comprising:
a. a first polynucleotide encoding a propeptide domain of a first polypeptide; and
b. a second polynucleotide encoding a second polypeptide of 2-50 amino acids having a modulating activity,
wherein said propeptide domain is a specific cell predominantly expressed protein precursor, and wherein said first polypeptide and said second polypeptide are not derived from the same gene and are operably linked.

2. The chimeric polynucleotide molecule of claim 1, wherein said propeptide domain comprises a signal peptide sequence and a protease/convertase motif.

3. The chimeric polynucleotide molecule of claim 2, wherein said signal peptide sequence and said protease/convertase motif are endogenous to said propeptide.

4. The chimeric polynucleotide molecule of claim 1, wherein said modulating peptide is an endogenous peptide.

5. The chimeric polynucleotide molecule of claim 1, wherein said modulating peptide is an exogenous peptide.

6. The chimeric polynucleotide molecule of claim 1, wherein said modulating peptide is a synthetic peptide.

7. The chimeric polynucleotide molecule of claim 1, wherein said propeptide domain is encoded by a polynucleotide sequence as set forth in SEQ ID NO:1 or SEQ ID NO: 2.

8. A delivery vector for expression of the chimeric polynucleotide of claim 1.

9. The delivery vector of claim 8, comprising a promoter polynucleotide for driving the expression of the chimeric polynucleotide of claim 1 predominantly in a specific cell.

10. The delivery vector of claim 9, wherein said specific cell is selected from the group consisting of a neural cell, a muscle cell, a liver cell, and a pancreas cell.

11. (canceled)

12. (canceled)

13. (canceled)

14. The delivery vector of claim 8, wherein the first polynucleotide sequence encoding the propeptide domain of the first polypeptide is selected from the group consisting of neurotrophic factors.

15. The delivery vector of claim 8, wherein the first polynucleotide sequence encoding the propeptide domain of the first polypeptide is selected from the group consisting of procollagens, is polypeptide derived from factor X, proinsulin, proglucagon, pro somatostatin, and proghrelin.

16. (canceled)

17. (canceled)

18. (canceled)

19. (canceled)

20. (canceled)

21. (canceled)

22. A cell comprising the delivery vector of claim 8, wherein said cell is selected from the group consisting of: sensory neurons, motor neurons, interneurons, neurons of the brain, astrocytes, microglia, ependymal cells, oligodendrocytes, Schwann cells, satellite cells, enteric glial cells, olfactory cells, and sheathing cells.

23. A cell comprising the delivery vector of claim 8, wherein said cell is selected from: (i) a cell selected from the group consisting of: red skeletal muscle cell, white skeletal muscle cell, intermediate muscle cell, nuclear bag cell, nuclear chain cell, satellite cell, heart muscle cell, nodal heart muscle cell, Purkinje fiber cell, smooth muscle cell, and myoepithelial cell; (ii) a cell selected from the group consisting of: hepatocyte, Kupffer cell, and liver stellate cell; (iii) a cell selected from the group consisting of: Alpha cell, Beta cell, Delta cell, Gamma cell, and Epsilon, cell; (iv) a cell selected from the group consisting of: Alpha cell, Beta cell, Delta cell, Gamma cell, and Epsilon cell.

24. (canceled)

25. (canceled)

26. A chimeric polypeptide encoded by the chimeric polynucleotide of claim 1.

27. A method for modulating a specific cell, comprising the step of contacting the specific cell with the chimeric polynucleotide of claim 1 thereby modulating the specific cell.

28. The method of claim 27, wherein said contacting the specific cell comprises the step of systemically administering to a subject in need thereof said chimeric polynucleotide, thereby producing post-translationally active modulating peptide only in a specific cell of the subject, thereby modulating only the specific cell of the subject.

29. A method for treating cell-specific-associated disease or disorder in a subject in need thereof, the method comprising administering to the subject the chimeric polynucleotide of claim 1 thereby treating cell-specific-associated disease in the subject.

30. The method of claim 29, wherein said cell-specific is selected from a neural cell, a muscle cell, a liver cell, and a pancreas cell.

31. (canceled)

32. (canceled)

33. (canceled)

34. The method of claim 30, wherein said disease or disorder is selected from the group consisting of: degenerative disease, developmental disease or cancer disease.


SEQUENCE LISTING (2 sequences)

SEQ ID NO: 1 - protein, 128 aa, Homo sapiens
MTILFLTMVISYFGCMKAAPMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRR

SEQ ID NO: 2 - protein, 121 aa, Homo sapiens
MSMLFYTLITAFLIGIQAEPHSESNVPAGHTIPQAHWTKLQHSLDTALRRARSAPAAAIAARVAGQTRNITVDPRLFKKRRLRSPRVLFSTQPPREAADTQDLDFEVGGAAPFNRTHRSKR